SEQUENCE LISTING (16 sequences)

SEQ ID NO: 1 - protein, 395 aa, Artificial Sequence
  TAZ
MNPSSVPHPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGGPAQQHAHLRQQSYDVTDELPLPPGWEMTFTATGQRYFLNHIEKITTWQDPRKVMNQPLNHVNLHPSITSTSVPQRSMAVSQPNLAMNHQHQQVVATSLSPQNHPTQNQPTGLMSVPNALTTQQQQQQKLRLQRIQMERERIRMRQEELMRQEAALCRQLPMETETMAPVNTPAMSTDMRSVTNSSSDPFLNGGPYHSREQSTDSGLGLGCYSVPTTPEDFLSNMDEMDTGENSGQTPMTVNPQQTRFPDFLDCLPGTNVDLGTLESEDLIPLFNDVESALNKSEPFLTWL

SEQ ID NO: 2 - protein, 272 aa, Artificial Sequence
  TAZ fragment
LPLPPGWEMTFTATGQRYFLNHIEKITTWQDPRKVMNQPLNHVNLHPSITSTSVPQRSMAVSQPNLAMNHQHQQVVATSLSPQNHPTQNQPTGLMSVPNALTTQQQQQQKLRLQRIQMERERIRMRQEELMRQEAALCRQLPMETETMAPVNTPAMSTDMRSVTNSSSDPFLNGGPYHSREQSTDSGLGLGCYSVPTTPEDFLSNMDEMDTGENSGQTPMTVNPQQTRFPDFLDCLPGTNVDLGTLESEDLIPLFNDVESALNKSEPFLTWL

SEQ ID NO: 3 - DNA, 1188 bp, Artificial Sequence
  TAZ
atgaatccgtcctcggtgccccatccgctcccgccgccagggcagcaagtcatccacgtcacgcaggacctggacaccgacctcgaagccctcttcaactctgtcatgaaccccaagcccagctcatggcggaaaaagatcctcccggagtccttctttaaggagcccgattccggctcgcactcgcgccaatccagcacagactcatcaggcggccacccggggcctcgactagctggcggcgcgcagcacgtccgctcgcactcgtcgcccgcatccctgcagctgggcaccggtgcgggagccgctggaggccctgcacagcagcatgcacatctccgccagcagtcctatgacgtgaccgacgagctgccgttgccccccgggtgggagatgaccttcacggccactggccagagatacttccttaatcacatagagaaaatcaccacatggcaagaccccaggaaggtgatgaatcagcctctgaatcatgtgaacctccacccgtccatcacttccacctcggtgccacagaggtccatggcagtgtcccagccgaatctcgcaatgaatcaccaacaccagcaagtcgtggccactagcctgagtccacagaaccacccgactcagaaccaacccacagggctcatgagtgtgcccaatgcactgaccactcagcagcagcagcagcagaaactgcggcttcagaggatccagatggagagagagaggattaggatgcgtcaagaggagctcatgaggcaggaagctgccctctgccgacagctccccatggaaaccgagaccatggcccctgtcaacacgcctgccatgagcacagatatgagatctgtcaccaacagtagctcagatcctttcctcaatggagggccctatcattcacgggagcagagcacagacagtggcctggggttagggtgctacagtgtccccacaactccagaagacttcctcagcaacatggacgagatggatacaggtgaaaattccggtcagacacccatgaccgtcaatccccagcagacccgcttccctgatttcctggactgccttccaggaacaaatgttgacctcgggactttggagtctgaagatctgatccctctcttcaatgatgtagagtctgctctgaacaaaagcgagccctttctaacctggctgtaa

SEQ ID NO: 4 - DNA, 819 bp, Artificial Sequence
  TAZ fragment
gagctgccgttgccccccgggtgggagatgaccttcacggccactggccagagatacttccttaatcacatagagaaaatcaccacatggcaagaccccaggaaggtgatgaatcagcctctgaatcatgtgaacctccacccgtccatcacttccacctcggtgccacagaggtccatggcagtgtcccagccgaatctcgcaatgaatcaccaacaccagcaagtcgtggccactagcctgagtccacagaaccacccgactcagaaccaacccacagggctcatgagtgtgcccaatgcactgaccactcagcagcagcagcagcagaaactgcggcttcagaggatccagatggagagagagaggattaggatgcgtcaagaggagctcatgaggcaggaagctgccctctgccgacagctccccatggaaaccgagaccatggcccctgtcaacacgcctgccatgagcacagatatgagatctgtcaccaacagtagctcagatcctttcctcaatggagggccctatcattcacgggagcagagcacagacagtggcctggggttagggtgctacagtgtccccacaactccagaagacttcctcagcaacatggacgagatggatacaggtgaaaattccggtcagacacccatgaccgtcaatccccagcagacccgcttccctgatttcctggactgccttccaggaacaaatgttgacctcgggactttggagtctgaagatctgatccctctcttcaatgatgtagagtctgctctgaacaaaagcgagccctttctaacctggctg

SEQ ID NO: 5 - DNA, 24 bp, Artificial Sequence
  primer
caaccaggaggagcgagacctccg

SEQ ID NO: 6 - DNA, 25 bp, Artificial Sequence
  primer
aggcgctgtgggatatgcattcact

SEQ ID NO: 7 - DNA, 20 bp, Artificial Sequence
  primer
cacctccacagcacagacag

SEQ ID NO: 8 - DNA, 20 bp, Artificial Sequence
  primer
accttggccatgtgattgtt

SEQ ID NO: 9 - DNA, 22 bp, Artificial Sequence
  primer
gtcaccaacagtagctcagatc

SEQ ID NO: 10 - DNA, 24 bp, Artificial Sequence
  primer
agtgattacagccaggttagaaag

SEQ ID NO: 11 - DNA, 22 bp, Artificial Sequence
  primer
aagcaggagtatgacgagtccg

SEQ ID NO: 12 - DNA, 22 bp, Artificial Sequence
  primer
cggaactaagtcatagtccgcg

SEQ ID NO: 13 - DNA, 24 bp, Artificial Sequence
  primer
ccccccaacacctgctgcctgagc

SEQ ID NO: 14 - DNA, 24 bp, Artificial Sequence
  primer
ggctcaggcagcaggtgttggggg

SEQ ID NO: 15 - DNA, 21 bp, Artificial Sequence
  primer
gaatcacatgtaatccactgg

SEQ ID NO: 16 - DNA, 21 bp, Artificial Sequence
  primer
cacaccaactgctgggtgcca

(19) United States
(12) Patent Application Publication
(10) Pub. No.: US 2011/0293599 A1
(43) Pub. Date: Dec. 1, 2011

(54) METHOD FOR MODULATING MUSCLE DIFFERENTIATION OR REGENERATION

(75) Inventor: Eun Sook Hwang, Seoul (KR)

(73) Assignee: EWHA UNIVERSITY - INDUSTRY COLLABORATION FOUNDATION, SEOUL (KR)

(21) Appl. No.: 13/086,593

(22) Filed: Apr. 14, 2011

(30) Foreign Application Priority Data
May 26, 2010 (KR) 10-2010-0049210

(51) Int. Cl. A61K 39/395; C12N 15/63; C12N 5/077; A61K 38/02; A61K 31/7088; A61P 21/00; C07H 21/00; A61K 38/17; A61P 9/00; A61P 25/00; A61P 9/10; G01N 33/68; C07K 14/47

(52) U.S. Cl. 424/130.1; 436/501; 435/320.1; 435/377; 435/375; 514/1.1; 514/44 R; 436/86; 530/350; 536/23.5; 514/21.2; 514/16.4; 514/17.7; 514/1.9

(57) ABSTRACT

Present invention relates to method for promoting or inhibiting muscle differentiation or regeneration using an enhancer or an inhibitor capable of modulating the binding of TAZ polypeptide with MyoD polypeptide; method for screening a substance capable of up- or down-regulating muscle differentiation or regeneration by use of interaction between TAZ polypeptide and MyoD polypeptide; an isolated peptide consisting of an amino acid sequence of SEQ ID NO. 2 which binds to and activates the MyoD polypeptide; a polynucleotide encoding the isolated peptide; and a pharmaceutical composition comprising the isolated peptide or the polynucleotide.

### PRIORITY INFORMATION

This application claims priority to Korean patent application No. 10-2010-0049210, filed on May 26, 2010, the entire contents of which are incorporated herein by reference.

### BACKGROUND OF INVENTION

1. Description of the Related Art

Muscle tissue in adult vertebrates regenerates from reserve myoblasts called satellite cells. Satellite cells are distributed throughout muscle tissue, and are mitotically quiescent in adult muscle when disease or injury is absent. Following muscle fiber injury or during the process of recovery from disease, satellite cells re-enter the cell cycle and proliferate 1) to fuse with existing muscle fibers, or 2) to differentiate into a new length of multinucleated myotube. The myoblasts ultimately yield replacement muscle fibers or fuse into existing muscle fibers, thereby increasing fiber girth by the synthesis of contractile apparatus components. This process is illustrated, for example, by the nearly complete muscle fiber regeneration that occurs in mammals following induced muscle fiber degeneration; the muscle progenitor cells proliferate and fuse together to regenerate muscle fibers.

Several growth factors regulating proliferation and differentiation of adult (and fetal) myoblasts have been found in vitro. Fibroblast growth factor is mitogenic for muscle cells, and also a potent inhibitor of muscle differentiation. Transforming growth factor-β (TGFβ) is a potent inhibitor of muscle differentiation, but shows no effect on myoblast proliferation. Insulin-like growth factors (IGFs) are active in stimulating both myoblast proliferation and differentiation in rodents.

TAZ (transcriptional coactivator with PDZ-binding motif) was originally identified as a 14-3-3-interacting cellular protein (1) and has been characterized to be a transcriptional coactivator that interacts with Runx2 through its WW domain and regulates Runx2-dependent osteoblast differentiation (2). TAZ also interacts with peroxisome proliferator-activated receptor-γ (PPARγ), an adipocyte-specific transcription factor, thus resulting in the inhibition of PPARγ-induced adipocyte differentiation (3). As expression levels of TAZ in mesenchymal stem cells are important for the 7fate of the cell decision into either osteoblasts or adipocytes, TAZ is suggested to function as a transcriptional modulator of mesenchymal stem cell differentiation (3).

In addition, TAZ interacts with several other transcription factors, including thyroid transcription factor-1 (TTF-1/Nkx2.1) (4, 5), T-box transcription factor (Tbx5) (6), Paired box gene 3 (Pax3) (7), Pax8 (4), Gli-Similar 3 (Glis3) (8), TEAD transcription factors (9, 10) and Smad2/3-4 complexes (11), and modulates their transcriptional activities and cellular functions.

Therefore, TAZ may be involved in a variety of biological functions such as kidney and lung formation (5, 8), cardiac and limb development (6), thyroid differentiation (4), embryonic stem cell self-renewal (11), and epithelial-mesenchymal transition and invasion of tumor cells (12).

Skeletal muscle possesses intrinsic repair potential derived from the satellite cells. In response to injury of adult skeletal muscle, quiescent satellite cells are activated and induce the expression of muscle regulatory factors (MRFs) including MyoD and the myocyte enhancer factor 2 (MEF2) families (13). Activated cells proliferate and express Myf5 and MyoD to form myoblasts (14) and undergo terminal differentiation and incorporation into muscle fibers (15). Myogenin expression is associated with terminal differentiation and fusion (16).

### SUMMARY OF INVENTION

An object of the present invention is to provide a method for promoting muscle differentiation or regeneration comprising the steps of administering an enhancer capable of inducing or promoting the binding of TAZ polypeptide with MyoD polypeptide to a subject or muscle cell thereof in need of muscle differentiation or regeneration

Another object of the present invention is to provide a method for inhibiting muscle differentiation or regeneration comprising the steps of administering an inhibitor capable of suppressing or inhibiting the binding of TAZ polypeptide with MyoD polypeptide to a subject or muscle cell thereof in need of inhibiting muscle differentiation or regeneration.

Another object of the present invention is to provide a method for screening a substance capable of up- or down-regulating muscle differentiation or regeneration by use of interaction between TAZ polypeptide and MyoD polypeptide.

Another object of the present invention is to provide an isolated peptide consisting of an amino acid sequence of SEQ ID NO. 2, a fragment thereof, or a variant of SEQ ID No. 2 having 60% or more homology therewith, wherein it binds to and activates the MyoD polypeptide; a polynucleotide encoding the isolated peptide; and a pharmaceutical composition comprising the isolated peptide or the polynucleotide encoding the same.

### DESCRIPTION OF PREFERRED EMBODIMENTS

In accordance with one aspect, the present invention provides a method for promoting muscle differentiation or regeneration, comprising the steps of administering an enhancer capable of inducing or promoting the binding of TAZ polypeptide with MyoD polypeptide to a subject or muscle cell thereof in need of muscle differentiation or regeneration.

In this regard, the method may further include the step of administering a TAZ polypeptide, a fragment thereof, or a polynucleotide encoding the same; a MyoD polypeptide, a fragment thereof, or a polynucleotide encoding the same; or a mixture thereof in addition to the enhancer, and the enhancer may show an inhibitory activity on an inhibitor that suppresses or inhibits the binding of TAZ polypeptide with MyoD polypeptide.

As used herein, the term “enhancer” refers to a substance capable of inducing or promoting the binding of TAZ polypeptide with MyoD polypeptide. The substance encompasses a protein, an antibody, an oligonucleotide, a peptide or a compound, but is not limited thereto.

As used herein, the term “muscle differentiation” refers to the induction of a muscle developmental program which specifies the components of the muscle fiber such as the contractile apparatus (myofibril).

As used herein, the term “muscle regeneration” refers to the process by which new muscle fibers form from muscle progenitor cells.

As used herein, the term “muscle cell” refers to any cell which contributes to muscle tissue, and encompasses myoblasts, satellite cells, myotubes, and myofibril tissues.

In accordance with another aspect, the present invention provides a method for inhibiting muscle differentiation or regeneration, comprising the steps of administering an inhibitor capable of suppressing or inhibiting the binding of TAZ polypeptide with MyoD polypeptide to a subject or muscle cell thereof in need of inhibiting muscle differentiation or regeneration. As used herein, the term “inhibitor” refers to a substance capable of suppressing or inhibiting the binding of TAZ polypeptide with MyoD polypeptide. The substance encompasses a protein, an antibody, an oligonucleotide, a peptide or a compound, but is not limited thereto.

The inhibitor inhibits the binding of TAZ polypeptide with MyoD polypeptide. Therefore, when a subject or muscle cell thereof is treated with the inhibitor, muscle cell differentiation or regeneration does not normally occur, and the differentiation or regeneration process may slow down or halt. These facts allow that the inhibitor can be utilized for intensive studies on muscle cell differentiation or regeneration, and also muscle differentiation or regeneration.

In accordance with still another aspect, the present invention provides a method for screening a substance capable of up- or down-regulating muscle differentiation or regeneration by use of interaction between TAZ polypeptide and MyoD polypeptide. The substance may be the above mentioned enhancer or inhibitor.

In this regard, the interaction may be a direct or indirect binding of TAZ polypeptide with MyoD polypeptide, but is not limited thereto.

The TAZ polypeptide or fragment thereof forms a heterodimer with MyoD or fragment thereof in muscle cells, and the heterodimer promotes myogenin expression, and the expressed myogenin subsequently induces muscle cell differentiation or regeneration. At this time, the binding of TAZ polypeptide or fragment thereof with MyoD or fragment thereof may be up-regulated by addition of a substance that is able to induce or promote the binding to enhance muscle differentiation or regeneration. On the contrary, the binding may be down-regulated by addition of a substance that is able to suppress or inhibit the binding to inhibit muscle differentiation or regeneration. Therefore, the present invention provides a method for screening a substance capable of up- or down-regulating the binding of TAZ polypeptide or fragment thereof with MyoD or fragment thereof.

The screening method is not particularly limited, but preferably includes the step of adding any substance, of which effect is unknown for muscle differentiation or regeneration in vivo or in vitro, to a TAZ and MyoD-existing system so as to detect effects of the substance. For example, the screening method may be developed to include the steps of adding the substance to a TAZ and MyoD-existing reactant under in vitro conditions, and then detecting effect of the substance on the binding of TAZ with MyoD by electrophoresis; the steps of expressing the substance in TAZ and MyoD-expressing muscle cells under in vivo conditions, and then analyzing muscle cell differentiation or regeneration so as to detect effect of the substance; or the steps of comparing the regeneration rates between the TAZ and MyoD-expressing injured muscle tissue and TAZ, MyoD, and the substance-expressing injured muscle tissue, and then detecting effect of the substance.

In accordance with still another aspect, the present invention provides an isolated peptide consisting of an amino acid sequence of SEQ ID NO. 1, a fragment thereof, or a variant of SEQ ID NO. 1 having 60% or more homology therewith, which binds to and activates the MyoD polypeptide. The present invention also provides an isolated peptide consisting of an amino acid sequence of SEQ ID NO. 2, a fragment thereof, or a variant of SEQ ID NO. 2 having 60% or more homology therewith, which binds to and activates the MyoD polypeptide. The isolated peptide consisting of an amino acid sequence of SEQ ID NO. 2 is an amino acid region at positions 124-395 of the TAZ polypeptide, which exhibits activities of inducing muscle differentiation or regeneration.

The homology of one amino acid sequence to another amino acid is defined as a percentage of identical or similar amino acids in two collated sequences. By term “similar amino acids” is meant that two compared amino acid residues in the collated sequences belong to the same group of amino acids. The sequence homology is calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90.

Further, the TAZ polypeptide is not particularly limited, but preferably consists of an amino acid sequence of SEQ ID NO. 1 or 2, and all peptides having the desired activity by one or more mutations in the amino acid sequence are also included in the scope of the present invention.

In accordance with still another aspect, the present invention provides a polynucleotide encoding the isolated peptide.

In this regard, the polynucleotide encoding the TAZ polypeptide is not particularly limited, but preferably consists of a nucleotide sequence of SEQ ID NO. 3. The polynucleotide encoding the isolated peptide is not particularly limited, but preferably consists of a nucleotide sequence (SEQ ID NO. 4) at 367-1185 of TAZ polypeptide-encoding polynucleotide. However, considering degeneracy of the genetic code and the variant peptide of the present invention, all polynucleotides having the desired activity by one or more mutations in the nucleotide sequence are also included in the scope of the present invention.

Furthermore, the polynucleotide may be included in an expression vector, and thus introduced into a host cell.

In accordance with still another aspect, the present invention provides a pharmaceutical composition comprising an isolated peptide or a polynucleotide encoding the same.

In this regard, the pharmaceutical composition is not particularly limited, but preferably used for inducing muscle differentiation or regeneration, or treating muscular diseases. Further, the muscular diseases are not particularly limited, but preferably include skeletal muscle diseases and disorders (e.g., myopathies, myoneural conductive diseases, traumatic muscle injury, or nerve injury), cardiac muscle pathologies (e.g., ischemic damage, congenital or traumatic disorders), or smooth muscle diseases or disorders (e.g., arterial sclerosis, vascular lesions, or congenital vascular diseases).

Moreover, the composition may further include a pharmaceutically acceptable carrier.

As used herein, the term “carrier” or “pharmaceutically acceptable carrier” means a diluent, adjuvant, excipient, or vehicle with which the substance is administered. Such pharmaceutical carriers include, but are not particularly limited to, any one of standard pharmaceutically carriers used in the known formulations such as sterile liquids, tablets, coated tablets and capsules. Typically, such carriers contain excipients such as polyvinylpyrrolidone, dextrin, starch, milk, sugar, certain types of clay, gelatin, stearic acid, talc, vegetable oils (vegetable oil, cottonseed oil, coconut oil, almond oil, or peanut oil), fatty acid esters such as fatty acid glyceride, mineral oil, Vaseline, animal fat, cellulose derivatives (e.g., crystalline cellulose, hydroxypropyl cellulose, hydroxypropyl methylcellulose, or methylcellulose) or other known excipients. These carriers may further include an antioxidant, a wetting agent, a viscosity stabilizer, a flavoring agent, a color additive and other ingredients. The composition containing the carrier may be formulated by the known method. Moreover, the composition may additionally contain a diluent, a dispersant, a surfactant, a binder, and a lubricant in order to formulate it into injectable formulations, such as aqueous solution, suspension, and emulsion, pills, capsules, granules and tablets. It is also possible to bind such carriers with a target organ-specific antibody or other ligands so that they may act specifically on a target organ.

The pharmaceutical composition of the present invention may be prepared in any form such as granule, powder, coated tablet, tablet, capsule, suppository, syrup, juice, suspension, emulsion, drop or injectable liquid formulation, and sustained release formulation of the active ingredient(s).

The pharmaceutical composition of the present invention may be administered to mammalian animals including human via various routes, for example, typical routes, such as intravenous, intraarterial, intraperitoneal, intramuscular, intrasternal, transdermal, intranasal, inhalational, local, rectal, oral, intraocular, and intradermal routes. The administration mode is not particularly limited, but the parenteral administration is preferred.

An effective dosage of the pharmaceutical composition of the present invention may be determined depending on various factors, including the kind and severity of diseases, the kind and content of an active ingredient and other components contained in the composition, the kind of a formulation, and patient's age, weight, general health condition, sex and diet, and administration time, administration route, the secretion % of the composition, administration period, and the kind of drugs used in combination. However, for better efficacy, the effective dosage of the pharmaceutical composition of the present invention may be administered at a daily dosage of about 1 mg/kg to 1 g/kg; and preferably about 0.01 mg/kg to 100 mg/kg once or several times.

As used herein, the term “treatment” means that the peptide and/or polynucleotide of the present invention are/is administered to a subject in need of treatment, for the purpose of reducing atrophy and degeneration of muscle cells.

As used herein, the term “therapeutically effective amount” or “effective amount” means that the amount of the substance is of sufficient quantity to produce therapeutic response. The therapeutic response may be any effective response to treatment, recognized by a user (that is, clinical investigator) such as through evaluation of symptoms and alternative clinical markers. Therefore, the therapeutic response may be alleviation in one or more symptoms of disease or disorder.

The effect of the pharmaceutical composition of the present invention may be visualized by growth of muscle. The growth of muscle may occur by the increase in the fiber size and/or by increasing the number of fibers. The growth of muscle as used herein may be measured by A) an increase in wet weight, B) an increase in protein content, C) an increase in the number of muscle fibers, or D) an increase in muscle fiber diameter. An increase in growth of a muscle fiber can be defined as an increase in the diameter where the diameter is defined as the minor axis of ellipse of the cross section. Each composition of the present invention increases the wet weight, protein content and/or diameter by 10% or more, more preferably by more than 50% and most preferably by more than 100%, relative to a similarly treated control animal (i.e., an animal with degenerated muscle tissue which is not treated with the muscle growth substance). In this regard, these percentages are determined relative to the basal level in a comparative untreated undiseased mammal or in the contralateral undiseased muscle when the substance is administered and acts locally.

Hereinafter, the present invention will be described in detail.

In this study, the inventors investigated the functions of TAZ and its regulatory mechanisms in muscle differentiation. While enforced TAZ expression in myoblasts enhanced myogenic gene expression and hastened multinucleated myofiber formation, reduction of TAZ delayed myogenic differentiation. Furthermore, TAZ increased myogenin expression through direct interaction with MyoD in the nucleus and enhancement of DNA binding activity of MyoD. These results imply that TAZ positively modulates myogenic differentiation via the activation of MyoD.

### Ectopic Expression of TAZ in Myoblasts Promotes Myogenic Differentiation.

TAZ expression in skeletal muscle progenitor cells and TAZ function in TEF-1-mediated muscle gene expression provoked us to explore the role of TAZ in muscle differentiation.

Since mouse-derived C2C12 myoblasts are able to differentiate into muscle cells when cultured in vitro in low mitogen medium, such as 2% horse serum, C2C12 cells were established that stably overexpress TAZ by retroviral transduction; we then induced myoblast differentiation in these cells. The level of TAZ expression was determined by quantitative real-time PCR, and was increased 2-3 fold in TAZ stable cells versus that in control cells (Supplementary FIG. 1A). Myoblast differentiation was observed in control C2C12 cells with cell morphological changes to a cylinder shape, but was found earlier and was profoundly increased in TAZ stable cells (FIG. 1A). MHC expression, as assessed by immunofluorescence staining, was advanced by ectopic expression of TAZ (FIG. 1B). Interestingly, multinucleated myofibers, which were formed by terminal differentiation and fusion, were clearly observed at day 3 after differentiation of TAZ-overexpressing cells (FIG. 1C). Investigation of the expression levels of muscle-specific gene markers demonstrated that enforced expression of TAZ in myoblast hastened myogenin induction and promoted MHC expression, but did not affect the expression of MyoD and MEF2C during myoblast differentiation (FIG. 1D). Quantitative real-time PCR also revealed that gene transcription levels of myogenin and muscle creatine kinase (MCK) were time-dependently increased in myogenesis and further augmented by increased expression of TAZ (FIG. 1E and 1F). These results suggest that enforced expression of TAZ promotes myogenic differentiation by increasing myogenic gene transcription.

### Reduced TAZ Expression Attenuates Myoblast Differentiation

Since the ectopic expression of TAZ enhanced myogenic differentiation, whether decreased TAZ may inhibit myoblast differentiation was questioned next. TAZ-knockdown C2C12 cells (shTAZ) were generated using small hairpin double-stranded RNA and found to express 3-fold less TAZ compared to control cells (Supple FIG. 1B). In response to differentiation into myocytes, MHC and myogenin expression were critically attenuated in TAZ-knockdown cells, as evaluated by immunofluorescence staining (FIG. 2A). In addition, protein expression levels of MHC and myogenin were diminished in myoblast differentiation of TAZ-knockdown cells. However, MyoD and MEF2C remained unchanged (FIG. 2B). Consistent with this, myogenin and MCK gene transcripts were markedly attenuated in TAZ-knockdown cells (FIGS. 2C and D), indicating that TAZ expression is important for modulating myoblast differentiation.

### TAZ Directly Regulates Gene Transcription of Myogenin and MCK.

Altered transcription levels of myogenin and MCK by TAZ expression inspired the examination of whether TAZ directly regulates gene transcription of myogenin and MCK. TAZ, a transcriptional regulator, cannot directly bind to a gene promoter or enhancer, but rather regulates activities of TAZ-interacting transcription factors (1). We transfected myogenin promoter-linked reporter gene (pMyo-luc) into TAZ or shTAZ cells and assayed the promoter activities after normalization with transfection efficiency. The myogenin promoter activity was increased in TAZ-overexpressing cells, but decreased in TAZ-knockdown cells (FIGS. 3A and B); MCK promoter activity was similarly modulated by TAZ. Myogenin transcription is regulated by MyoD-mediated chromatin remodeling and also positively autoregulated was tested. Thus, the effects of TAZ on the activity of MyoD or myogenin in the activation of myogenin promoter activity. Myogenin promoter activity was increased by MyoD and synergistically inflated by co-expression of TAZ (FIG. 3C). Enforced expression of myogenin also increased its own promoter activity by 5-fold, but did not show any cooperative activity with TAZ expression. In addition, MCK was also increased by either myogenin or MyoD. TAZ significantly enhanced the MyoD-induced MCK promoter activity, but did not affect myogenin-induced expression (FIG. 3D), suggesting that TAZ selectively promotes MyoD-mediated gene transcription. Electrophoretic mobility shift assays additionally revealed that TAZ increased the DNA binding activity of MyoD to the myogenin promoter (FIG. 3E). These results strongly suggest that TAZ increases MyoD-mediated gene transcription by enhancing the DNA binding activity of MyoD.

TAZ Physically Associates with MyoD.

The cooperative function of TAZ on MyoD activity and the structural features of TAZ protein prompted the examination of the physical association of TAZ and MyoD. Since WW and coiled-coil domains of TAZ are involved in protein-protein interactions, Flag-tagged TAZ and MYC-tagged MyoD was overexpressed in 293T cells for an in vitro interaction study.

Immune complexes of TAZ protein co-precipitated MyoD (FIG. 4A), suggesting a strong physical interaction between TAZ and MyoD. To identify the TAZ domain required for interaction with MyoD, TAZ truncation mutants were generated and cotransfected with MyoD into 293T cells. While full-length TAZ and ΔN1 (aa 124-395 TAZ) interacted with MyoD with high affinity, ΔN2 (aa 164-395 TAZ), which lacked the WW domain, failed to interact with MyoD (FIG. 4B). Subsequent binding assay using NT TAZ (aa 1-163) provided that the WW domain of TAZ was required for interacting with MyoD (Suppl. FIGS. 2A and B).

Conversely, MyoD truncations were produced and coexpressed with TAZ. While NT (MyoD aa 1-102) MyoD had no interaction with TAZ, CT MyoD (aa 162-318) selectively interacted with TAZ (FIG. 4C). In vitro binding studies using MyoD truncations further suggested that CT MyoD may be involved in the interaction with TAZ. To assure the interaction between TAZ and MyoD in vivo, an endogenous co-immunoprecipitation assay was conducted using TAZ stable cells expressing Flag-tagged TAZ. TAZ immune complexes were immunoprecipitated with anti-Flag Ab and concomitantly pulled down endogenous MyoD protein during myogenic differentiation (FIG. 4D), implying that the interaction between endogenous MyoD and TAZ may occur specifically during myogenic differentiation.

TAZ Interacts with MyoD in the Nucleus upon Myogenic Differentiation Stimulation.

Investigation of the subcellular localization of MyoD and TAZ was made next. Confocal microscopic observation revealed that TAZ was expressed in both the nucleus and the cytosol of C2C12 cells under growing conditions with 10% FBS (FIG. 5A). However, most TAZ protein was translocated to the nucleus under differentiation conditions, which were confirmed by immunofluorescence staining and immunoblotting (FIGS. 5A and B). In addition, TAZ was expressed with punctate nuclear distribution and co-localized with MyoD in the nucleus at day 2 post-differentiation (FIG. 5C). Investigation of whether TAZ and MyoD complex was positioned on the myogenin promoter during myogenic differentiation was made next. Chromatin immunoprecipitation experiments revealed that MyoD bound to the E-box region of myogenin promoter and TAZ protein was additively associated with a region of MyoD binding site in the myogenin promoter (FIG. 5D).

In addition, MyoD interaction with the myogenin promoter was clearly attenuated by reduced TAZ expression in TAZ-knockdown cells (FIG. 5E). These results suggest that TAZ may facilitate the binding activity of MyoD to the myogenin promoter via their physical interaction.

TAZ Enhances Myogenic Differentiation Through Cooperative Functional Interaction with MyoD.

To verify the cooperative synergy between TAZ and MyoD in myogenic differentiation, MyoD and TAZ were comparably expressed following retroviral transduction (FIG. 6A). Introduction of MyoD itself into C3H10T1/2 cells induced the expression of myogenin and MHC, deriving myogenic differentiation (FIG. 6B). TAZ expression alone had no effect on myogenic differentiation, whereas concurrent expression of TAZ and MyoD cooperatively enhanced MyoD-derived myogenic differentiation (FIG. 6B). Quantitative analysis of MHC-positive and myogenin-positive cell populations confirmed the cooperative effects of TAZ and MyoD on the expression of MHC and myogenin (FIG. 6C). Moreover, TAZ synergistically augmented protein expression of MHC and myogenin induced by MyoD (FIG. 6D), implying that TAZ functions as a cooperative regulator of MyoD for inducing myogenic differentiation.

### Myogenic Differentiation of MyoD-Introduced MEFs is Modulated by TAZ.

Investigation of whether TAZ deficiency affects myogenic differentiation from MEFs was made next. WT and KO MEFs established from the embryos of WT and TAZ KO mice were transduced with either control or MyoD expression vector. Established MEFs clones were then cultured under myogenic differentiation conditions. Quantitative real-time PCR results confirmed that TAZ was not expressed in KO MEFs (FIG. 7A) and that enforced MyoD expression was comparably increased in WT and KO MEFs (FIG. 7B). Ectopic expression of MyoD increased myogenin mRNA level in WT MEFs, whereas TAZ deficiency interfered with myogenin gene transcription induced by MyoD (FIG. 7C). MCK expression was also impaired in TAZ KO MEFS, as compared with WT cells (FIG. 7D). To ask whether restored TAZ expression can retrieve the myogenic activity of MyoD in TAZ KO MEFs, TAZ was introduced into TAZ KO MEFS in concert with MyoD with an equal expression level (FIGS. 7E and F). TAZ restoration increased the amounts of myogenin and MCK (FIGS. 7G and H), indicating that TAZ is essential for the promotion of MyoD-induced myogenic differentiation at MEFs.

### Injury-Induced Muscle Regeneration is Mediated by the Induction of Endogenous TAZ Expression.

Because TAZ cooperatively increased MyoD-dependent myogenin expression and myogenic differentiation, the in vivo relevance of TAZ function in muscle differentiation was examined using a local freeze injury-induced in vivo myogenesis model. in vivo muscle regeneration was induced by direct application of a pre-cooled metal probe to the muscle for 5 s and increased numbers of mononuclear cells called satellite cells were observed within interstitial positions at day 13 post-injury compared to day 2 (FIG. 8A), suggesting an active in vivo muscle differentiation at day 13 post-freeze injury. Immunostaining of TAZ protein revealed increased expression of endogenous TAZ in regenerating muscle after injury (FIG. 8B). Additional immunoblotting assays verified that TAZ expression was significantly increased at day 13, while MyoD expression remained unchanged between days 2 and 13 (FIG. 8C). Compatible with TAZ induction, myogenin expression was increased during active muscle differentiation (FIG. 8C), supporting the in vivo activation of myogenin by TAZ induction.

TAZ may Interact with the Complex of MyoD and p300/CBF to Activate MyoD-Dependent Gene Transcription.

Skeletal muscle differentiation is one of the best known examples of endogenous regeneration and repair systems that replace dead or damaged muscle cells. Skeletal muscle is regenerated from mitotically quiescent satellite cells located in adult skeletal myofibers. Satellite cells rapidly proliferate upon activation signals such as muscle damage or loss and function as myogenic precursors or myoblasts. Myoblasts further undergo myogenic differentiation and fuse to give rise to multinucleated myotubes and skeletal myofibers upon the synthesis of muscle specific factors (actin, myosin, tropomyosin, creatine phosphate kinase). Myogenic differentiation proceeds with the sequential activation of muscle specific transcription factors and recruitment of transcriptional co-activators(15). In particular, MyoD is exclusively expressed in muscle precursor and muscle cells and plays a key regulatory role in the activation of muscle-specific gene expression. While ectopic introduction of MyoD induces trans-differentiation of fibroblasts into muscle cells, mice lacking MyoD fails to generate skeletal muscle(16). MyoD forms heterodimers with many other transcription factors and cooperatively recruits transcriptional co-regulators for its target gene expression. The interaction between p300/CBP and MyoD is required for the expression of muscle-specific genes.

Notably, TAZ interacts with p300/CBP(6), which also interacts with MyoD, suggesting a simple configuration in which the triple complex may function on the myogenin promoter (FIG. 9).

Increased Expression of Endogenous TAZ is Critical for the Robust Myogenin Expression and the Muscle Differentiation in vivo.

Direct injury to the muscle such as puncturing, cutting and freezing induces in vivo muscle regeneration, which is prominently driven by the proliferation and terminal differentiation of infiltrated satellite cells. Upon muscle injury, muscle undergoes a series of muscle degeneration, inflammation, regeneration and fibrosis.

While muscle degeneration and inflammation occur in the first few days post-injury, muscle regeneration occurs 7 to 10 days, peaks at 2 weeks and then disappears at 3 to 4 weeks after injury. It was observed that endogenous TAZ expression, not MyoD, is substantially increased at day 13 post-freeze injury, which coincides with the peak of muscle regeneration. Instantaneous increases of myogenin expression with TAZ expression strongly suggest that TAZ expression is crucial for robust myogenin expression in muscle differentiation in vitro and in vivo.

Therefore, induction of endogenous TAZ expression in mesenchymal stem cells could be privileged to induce adult muscle differentiation and repair.

The present study demonstrates that TAZ associates with MyoD and activates MyoD-dependent myogenic gene transcription, thereby promoting myogenic differentiation in vitro. In addition, instantaneous increases of myogenin expression with TAZ expression are prominent during in vivo muscle regeneration. These results strongly suggest that TAZ expression is crucial for robust myogenin expression in muscle differentiation in vitro and in vivo.

### DESCRIPTION OF DRAWINGS

FIG. 1. Enforced TAZ expression in myoblasts hastens myogenic differentiation. Control C2C12 cells (CON) and selected stable C2C12 cells (TAZ) that overexpress TAZ were grown to confluence and fed with differentiate medium.
(A) Morphological alterations during myogenic differentiation were observed under the microscope. Scale bar indicates 100 μm.
(B) Cells were fixed at the indicated time points and incubated with anti-MHC antibody, followed by staining with Alexa Fluor 488-conjugated anti-rabbit IgG and confocal microscopic observation (Scale bar, 100 μm).
(C) Immunofluorescence staining of differentiated CON and TAZ cells was conducted using with anti-MHC Ab. Images were pictured with 50 μm scale bar.
(D) Whole cell extracts were prepared from the differentiating cells and resolved by SDS-PAGE. Protein blots were incubated with antibodies against TAZ, MHC, myogenin, MyoD, MEF2C and β-actin.
(E and F) Total RNA was collected and reverse transcribed into cDNA. Relative gene transcription levels of myogenin (E) and MCK (F) were determined after normalization to β-actin level in real-time PCR. *, P<0.05; **, P<0.005.

FIG. 2. Reduction of endogenous TAZ delays myoblast differentiation. TAZ-knockdown C2C12 cells (shTAZ) were established and used for myogenic differentiation.
(A) Microscopic observation was conducted at days 2 and 3 after differentiation. Cells were incubated with anti-MHC or anti-myogenin antibodies and observed under a confocal microscope.
(B) Whole cell extracts were analyzed by SDS-PAGE and immunoblot assay using TAZ, MHC, myogenin, MyoD, MEF2C and β-actin antibodies. (C and D) The levels of mRNA were determined by quantitative real-time PCR and relative expression levels were presented after normalization to β-actin level. **, P<0.005; ***, P<0.0005.

FIG. 3. TAZ increases DNA binding and transcriptional activity of MyoD. The myogenin promoter-linked reporter gene (pMyo-luc) was transfected into control (CON) and TAZ stable cells (A) or control knockdown (shCon) and TAZ-knockdown (shTAZ) cells. Promoter activity was calculated from luciferase activity normalized to galactosidase activity and expressed as fold-change compared to control. Data are given as means ±SEM of three independent experiments. *, P<0.05.
(C-D) Parental C2C12 cells were transfected with plasmids that express MyoD, myogenin, or MEF2C with (+TAZ) or without (−TAZ) TAZ expression vector concomitant with either myogenin promoter reporter gene (pMyo-luc, C) or MCK promoter reporter gene (pMCK-luc, D). Three independent experiments were conducted and results are reported as mean ±SD. *, P<0.05; **, P<0.005. (E) 293T cells were transfected with MyoD and/or TAZ expression vectors. The nuclear proteins were harvested and incubated with radiolabeled E-box oligonucleotide as a probe. The DNA-protein complexes were resolved by native-PAGE and exposed to radiographic film.

FIG. 4. TAZ physically associates with MyoD on the myogenin promoter.
(A) 293T cells were transfected with Flag-tagged TAZ and/or MyoD expression plasmids. Immune complexes precipitated using Flag-M2 agarose beads were resolved by SDS-PAGE and immunoblotted with MyoD Ab. The expression levels of MyoD and TAZ were determined in whole cell extracts.
(B) TAZ truncations (N1 and N2) were generated and cotransfected with MyoD into 293T cells. Immunoprecipitation was conducted using Flag-M2 beads and analyzed by immunoblotting of MyoD.
(C) MyoD full length (FL) and truncations (NT and CT) were constructed in Myc-tagged expression vector and cotransfected with Flag-TAZ. Whole cell extracts and Flag-M2 immunoprecipitates were resolved by SDS and subsequent immunoblotting.
(D) Confluent TAZ stable cells that express Flag-tagged TAZ were cultured in differentiation medium for 2 days. TAZ-interacting proteins were precipitated by incubating with Flag-M2 agarose beads. Immune complexes were analyzed by immunoblotting using anti-MyoD antibody.

FIG. 5. TAZ translocates to the nucleus and cooperatively binds to the myogenin promoter in myogenic differentiation. (A) C2C12 cells were cultured in growth medium (GM) or differentiated under differentiation conditions (DM) and stained with anti-TAZ Ab and Alexa Fluor 555 anti-rabbit IgG after fixation. Nuclei were stained with DAPI. (B) Nuclear (Nuc) and cytosolic (Cyt) proteins were harvested from cells in growing (GM) or differentiation conditions (DM) and used for immunoblotting of TAZ and MyoD. Anti-OCT1 Ab was used as a positive control for nuclear protein. Ns indicates non-specific band.
(C) C2C12 cells were differentiated for 2 days and stained with anti-TAZ and anti-MyoD antibodies. Cells were then incubated with secondary IgG linked with Alexa Fluor 555 (red, TAZ) or Alexa Fluor 488 (green, MyoD) and observed with the confocal microscope. Scale bar indicates 10 μm.
(D) Soluble chromatins were prepared from control and TAZ stable cells that were induced to myogenic differentiation for 2 days, and incubated with control IgG, anti-Flag or anti-MyoD antibody. DNA was eluted from the immune complexes and used for PCR with primers that recognize the MyoD binding site in the myogenin promoter. Equal amounts of input chromatin were used for the analyses.
(E) TAZ and shTAZ stable cells were treated for myogenic differentiation for 2 days and harvested for chromatin immunoprecipitation assays. Soluble chromatins were prepared and precipitated using control IgG or anti-MyoD antibody. The MyoD binding site in the myogenin promoter was amplified by PCR.

FIG. 6. TAZ cooperatively promotes MyoD-mediated trans-differentiation of fibroblasts into myoblasts.
(A) C3H10T1/2 cells were infected with MyoD- and TAZ-expressing viruses. Protein expression was determined in whole cell extracts.
(B) C3H10T1/2 cells introduced with MyoD and/or TAZ were differentiated for 7 days in differentiation medium. MHC and myogenin expression were analyzed by immunofluorescence staining. Scale bar indicates 100 m.
(C) Fluorescence-positive cells were counted and statistical significance was determined. *, P<0.05.
(D) Whole cell extracts were prepared from the cells during myogenic differentiation and used to determine the expression levels of MHC and myogenin by immunoblotting.

FIG. 7. TAZ facilitates MyoD-induced myogenic differentiation of MEFs.
(A-B) MEFs isolated from WT and TAZ KO mice were infected with MyoD-expressing viruses and subjected to differentiate under myogenic differentiation conditions for 4 days. Total RNA was isolated and used for reverse transcription and real-time PCR analysis of TAZ (A), MyoD (B), myogenin (C) and MCK (D). (E-H) TAZ KO MEFs were retrovirally transduced with MyoD, TAZ or MyoD plus TAZ. Gene transcripts of MyoD (E), TAZ (F), myogenin (G) and MCK (H) were determined by real-time PCR. Relative expression level was determined after normalization with β-actin level. nd, not determined; *, P<0.05; **, P<0.005.

FIG. 8. Endogenous TAZ is increased in muscle differentiation to induce myogenin expression in vivo.
(A) C57BL/6 mice (n=5 for each) were injured by applying a cold metal probe into muscle. Injured muscle was harvested at days 2 and 13 post-injury and fixed in 4% paraformaldehyde. Muscles were sectioned in a microtome cryostat and stained with hematoxylin and eosin.
(B) Muscle sections obtained on days 2 and 13 were stained with anti-TAZ Ab and subsequently developed with a DAB staining kit. Scale bar indicates 500 μm.
(C) Whole cell extracts were harvested from the injured muscles at days 2 and 13 and analyzed by immunoblotting of TAZ, MyoD and myogenin.

FIG. 9. Schematic illustration of TAZ functional mechanism in myogenic differentiation.
TAZ cooperatively interacts with MyoD and transactivates gene transcription of myogenin and MCK. In addition, increased level of myogenin subsequently boosts myogenic gene expression and induces terminal differentiation. BTCs indicate the basal transcription complexes. MRFs mean myogenic regulatory factors.

FIG. 10 shows expression levels of TAZ in TAZ and shTAZ stable cells, in which Mock (CON) and TAZ-overexpressing (TAZ) C2C12 cells(10a), or control knockdown (shCon) and TAZ-knockdown (shTAZ) cells(10b) were differentiated under myogenic differentiation conditions and harvested at the indicated time points, total RNA was collected and used for reverse transcription and real-time PCR, and relative expression levels of TAZ were determined by normalization to the levels of β-actin (*, P<0.05.).

FIG. 11 shows N-terminus of TAZ involved in the interaction with MyoD, in which 11a is a schematic diagram of TAZ full length(FL)(1-395 aa) and TAZ NT (TAZ 1-163 aa) and 11b is 293T cells transfected with TAZ FL and NT with or without MyoD, and immunoprecipitation was conducted using Flag-M2 agarose beads and washed with lysis buffer, and immune complexes and whole cell extracts were resolved by SDS-PAGE and immunoblotted with anti-Flag and anti-MyoD Ab.

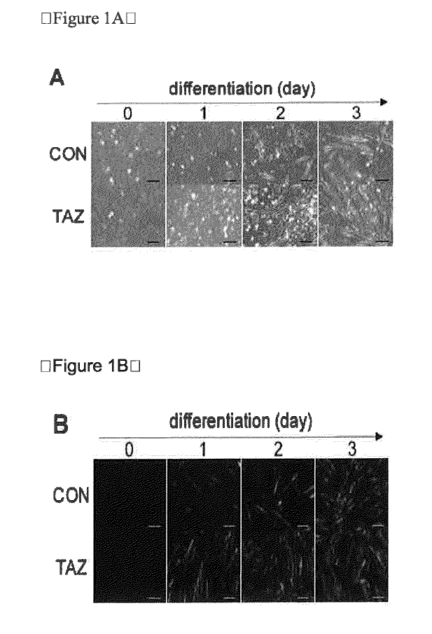

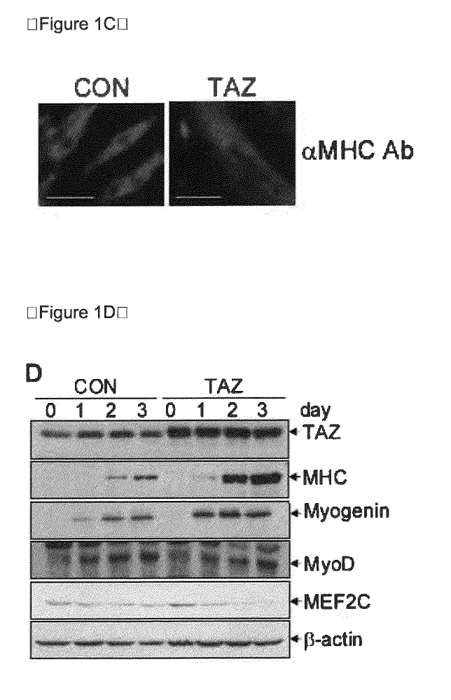

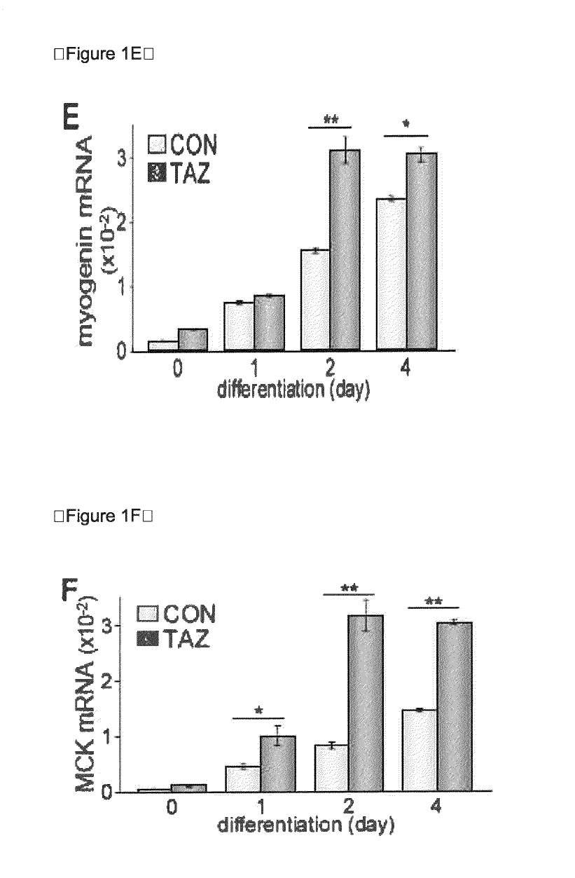

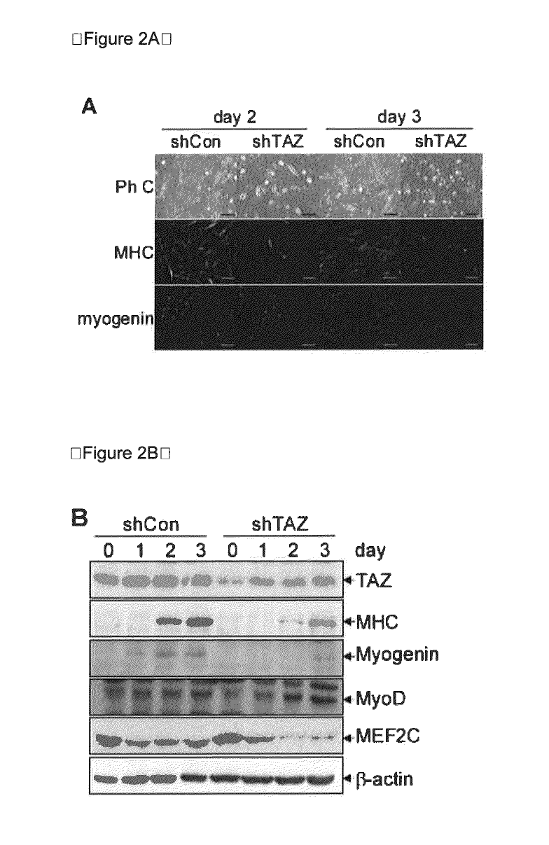

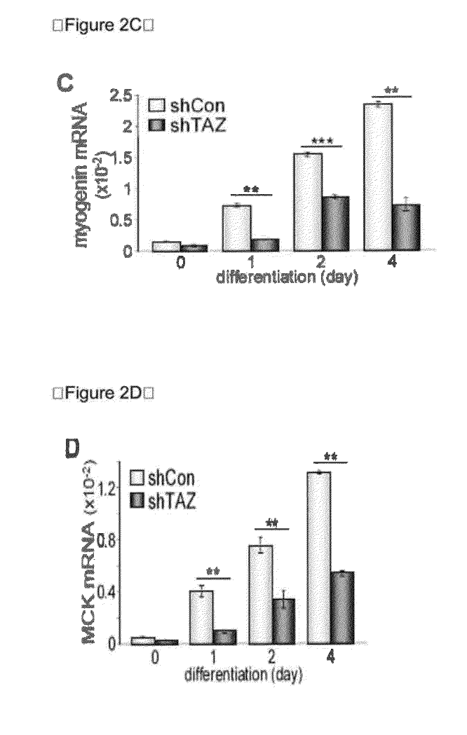

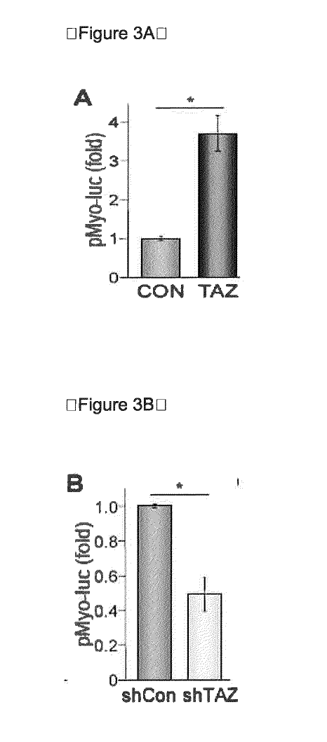

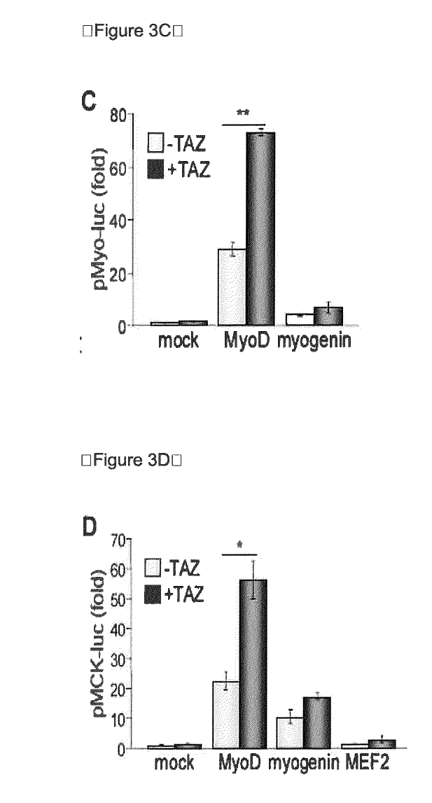

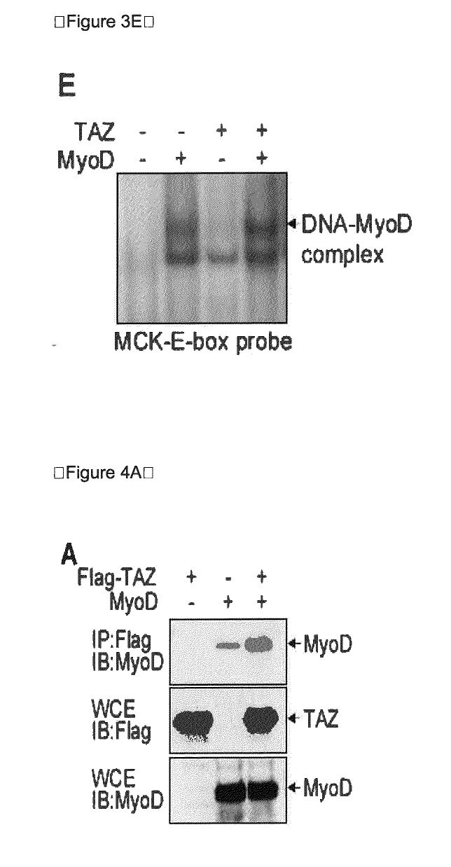

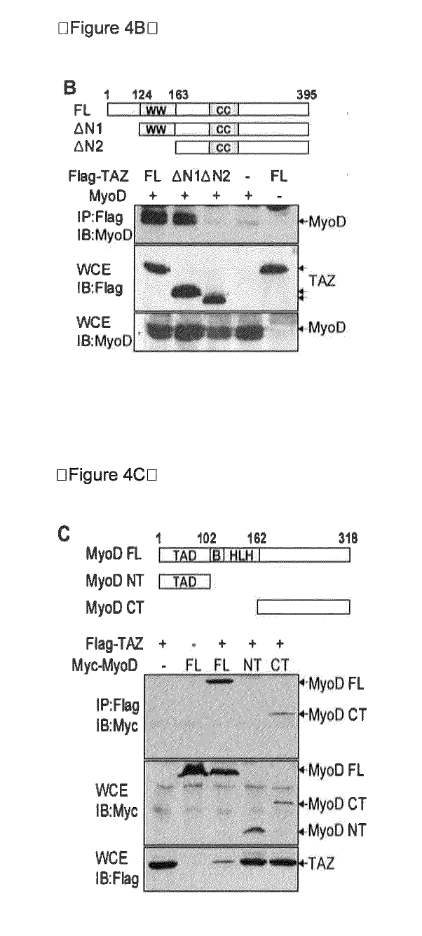

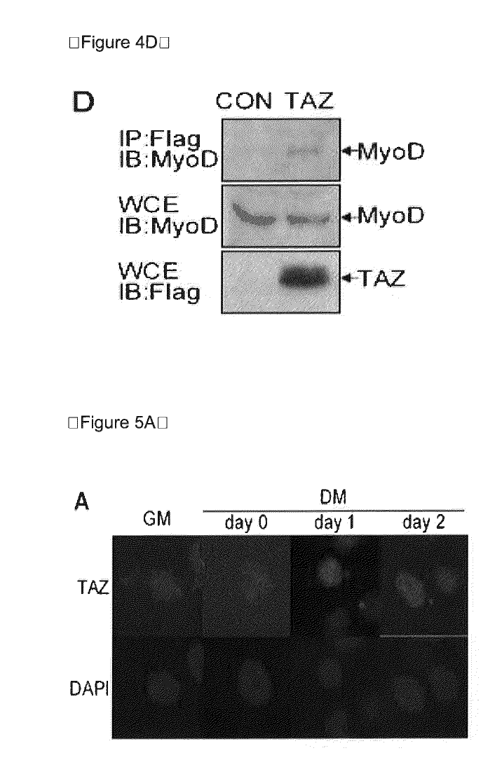

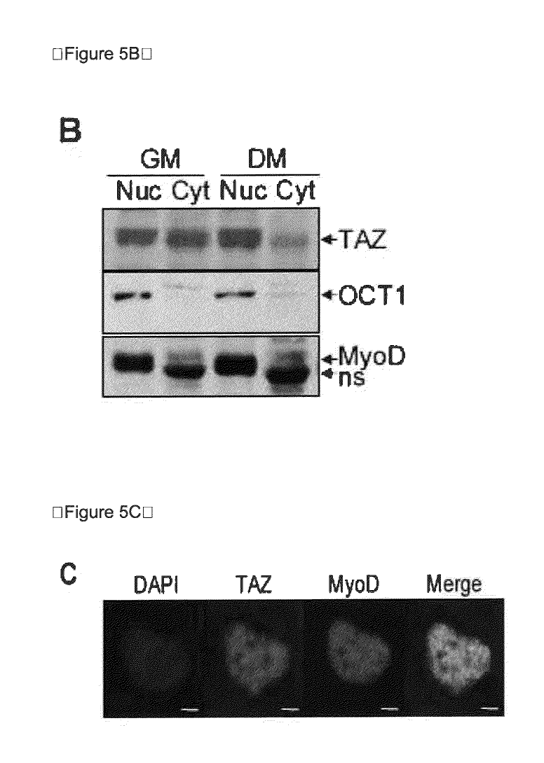

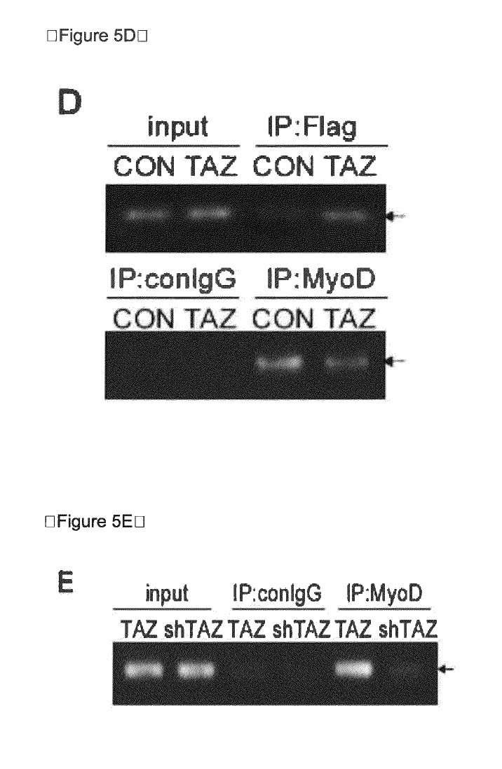

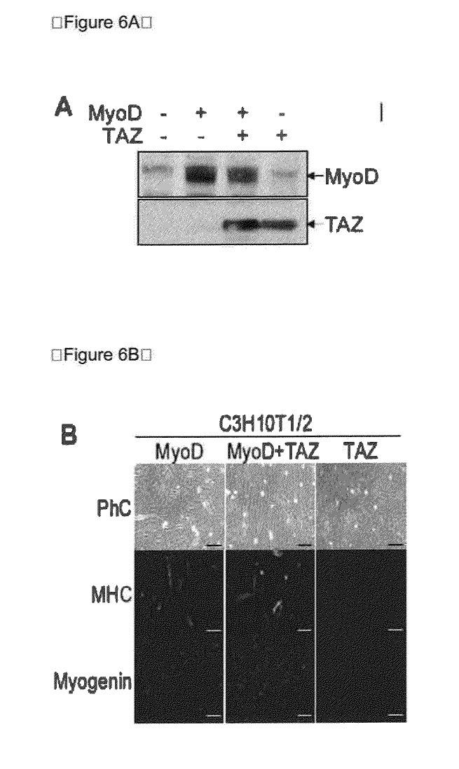

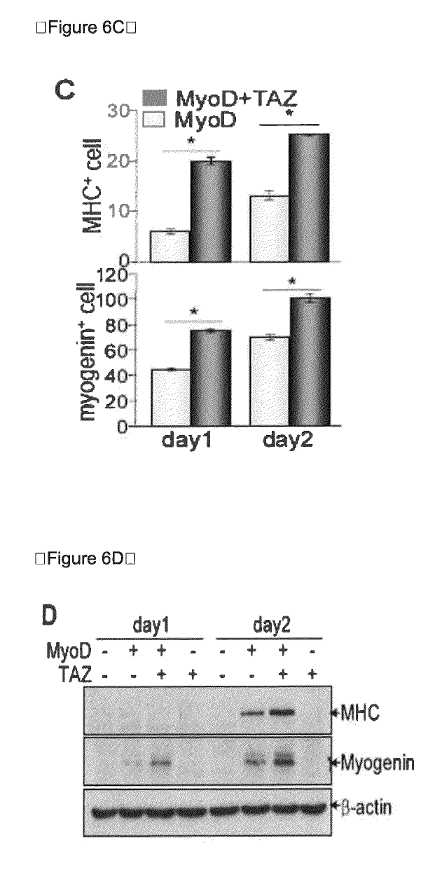

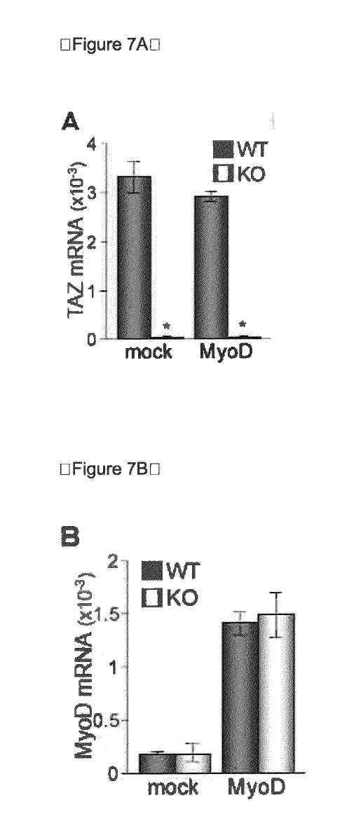

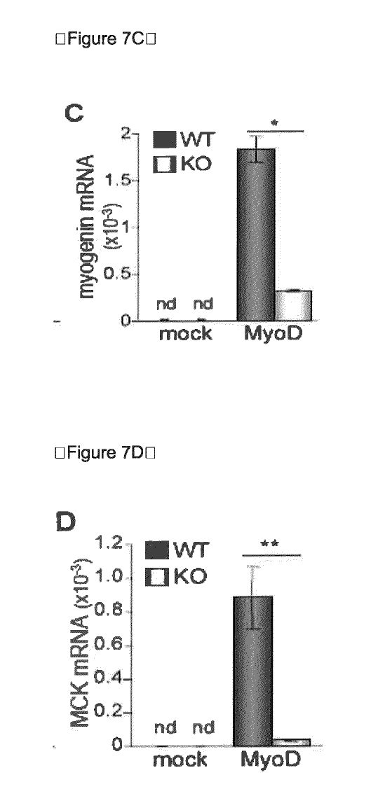

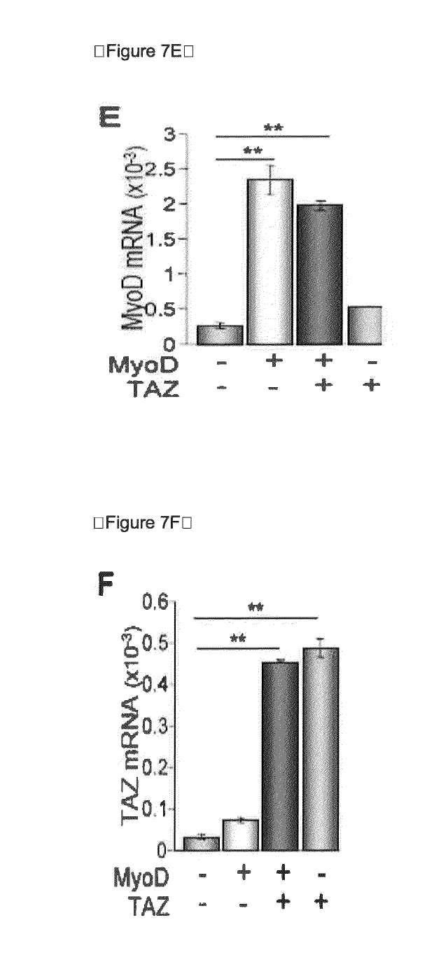

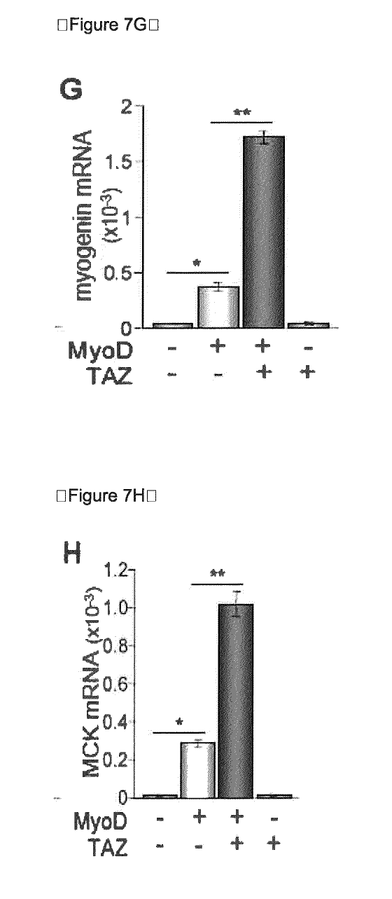

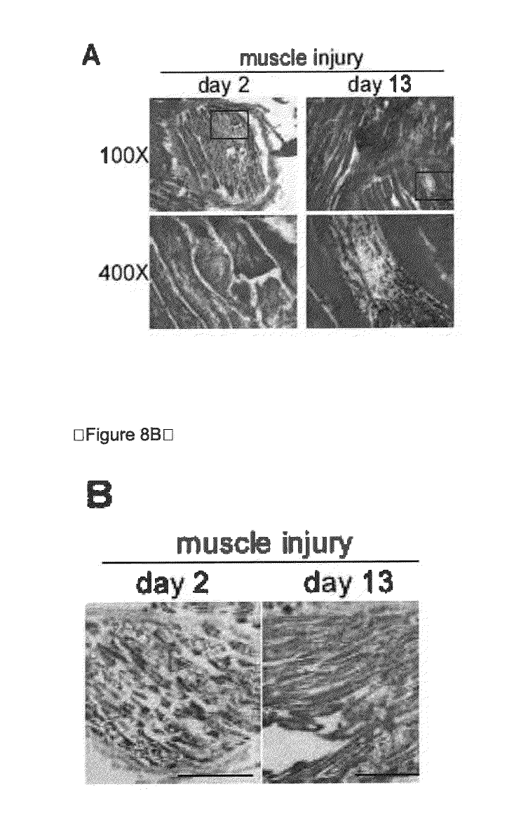

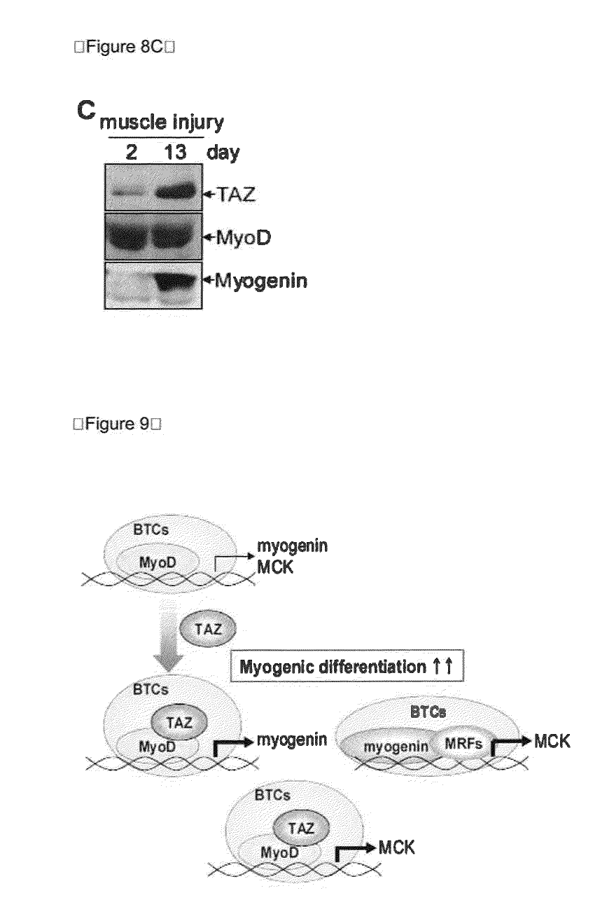

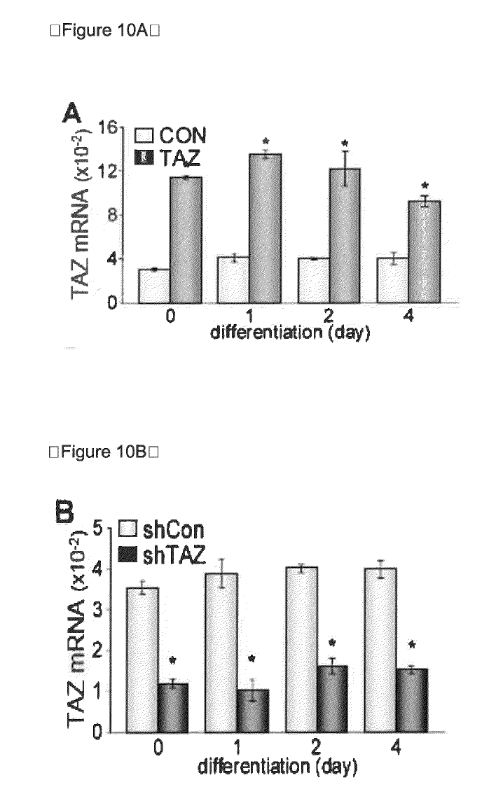

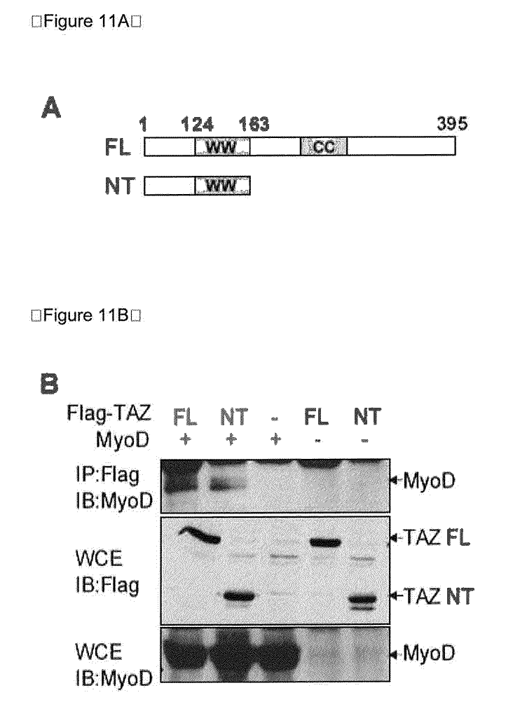

### MODE FOR INVENTION

Hereinbelow, the present invention will be described in more detail with reference to non-limitative Examples.

However, these Examples are for illustrative purposes only, and the invention is not intended to be limited by these Examples.

MyoD expression vectors were generated by inserting cDNAs corresponding to the full length 318 aa (FL), N-terminal 102 aa (NT), N-terminal 162 aa (NTBH), or C-terminal 160 aa (CT) into pCMV-Myc vector (Invitrogen, Carlsbad, Calif., USA). Full-length MyoD cDNA was also integrated into pBabe-puro vector (pBP, Cell Biolabs, Inc. San Diego, Calif., USA) for retroviral transduction. TAZ expression vectors (TAZ FL, N1, N2, and NT) and TAZ knockdown vector (pSRP-TAZ) (3) were used for the transfection of cells.

### EXAMPLE 1

### Cell Culture and Myogenic Differentiation

0080 C2C12 or C3H10T1/2 cells were obtained from American Type Culture Collection (Manassas, Va., USA) and maintained in growth medium containing 10% fetal bovine serum (FBS, HyClone, Logan, Utah, USA) in Dulbecco's modified Eagle's medium (DMEM, Invitrogen, Carlsbad, Calif., USA). For myogenic differentiation, cells were grown to confluence for 2 days and then incubated with differentiation medium consisting of 2% horse serum (Invitrogen) in DMEM. Cells Differentiation medium was replenished every 2 days. 293T cells were cultured in DMEM supplemented with 10% FBS. Mouse embryonic fibroblasts (MEF) were isolated from WT and TAZ KO mice and cultured in the complete DMEM as previously described (3). For myogenic differentiation, MyoD-introduced MEFs were cultured to confluence for 2 days and were then replenished with DMEM containing 2% horse serum every other day.

### EXAMPLE 2

### Generation of Stable COell Lines by Retroviral Transduction

Phoenix cells were cultured and transfected with the retroviral vectors pBabe-puro (pBP, Cell Biolabs, Inc., San Diego, Calif., USA), pBP-TAZ (TAZ overexpression vector), pSRP or pSRP-TAZ vector using the calcium phosphate transfection method (3). The cells were refreshed and transferred to a 32° C. incubator for another 24 h. The viral supernatants were collected and aseptically filtered through a 45-m pore cellulose acetate membrane (Invitrogen). C2C12 or C3H10T1/2 cells were incubated with viral supernatants and polybrene (8 /ml, Sigma-Aldrich, St. Louis, Mo., USA) for 48 h, and cell clones were established by limiting dilution protocol in the presence of 2.5 μg/ml puromycin (Sigma-Aldrich) for 7 days. MEFs were also transduced with viral supernatants expressing MyoD, TAZ or MyoD plus TAZ and were subsequently selected in the presence of puromycin for 7 days.

### EXAMPLE 3

### Reporter Gene Assay

C2C 12 cell clones were transfected with the myogenin promoter-linked reporter gene (pMyo-luc) using calcium phosphate method. 293T cells were transfected with expression plasmids for MyoD, myogenin, MEF2 or TAZ and the reporter gene pMyo-luc or the MCK promoter-linked reporter gene (pMCK-luc). pCMV-β vector (Invitrogen) encoding β-galactosidase was co-transfected as an internal control for normalization of transfection efficiency. Following transfection, growth medium was replaced with differentiation medium and cells were incubated for an additional 24 h. Cells were harvested and assayed using the luciferase assay kit (Promega, Madison, Wisc., USA) and the galactosidase assay kit, Galacto-Light™ (TROPIX, Bedford, Mass., USA).

### EXAMPLE 4

### Real-Time PCR Analysis

Real-time PCR for quantitative measurement of gene transcription levels was performed as described previously. Briefly, cells were harvested and incubated with TRIzol reagent (Gibco-BRL, Invitrogen). Total RNA was prepared and subjected to reverse transcription using Superscript II (Invitrogen). Real-time PCR reactions were performed in a mixture containing 1/20 volume of cDNA preparation, 1×SYBR Green pre-mix buffer (Perkin-Elmer Applied Biosystems, Foster City, Calif., USA) and specific primers. Primers were as follows:

myogenin-FWD,
(SEQ ID No. 5)
5′-CAACCAGGAGGAGCGAGACCTCCG-3′;
myogenin-REV,
(SEQ ID No. 6)
5′-AGGCGC TGTGGGATATGCATTCACT-3′;
MCK-FWD,
(SEQ ID No. 7)
5′-CACCTCCACAGCACAGACAG-3′;
MCK-REV,
(SEQ ID No. 8)
5′-ACCTTGGCCATGTGATTGTT-3′;
TAZ-FWD,
(SEQ ID No. 9)
5′-GTCACCAACAGTAGCTCAGATC-3′;
TAZ-REV,
(SEQ ID No. 10)
5′-AGTGATTACAGCCAGGTT AGAAAG-3′
and
β-actin-FWD,
(SEQ ID No. 11)
5′-AAGCAGGAGTATGACGAGTCCG-3′;
β-actin-REV,
(SEQ ID No. 12)
5′-CGGAACTAAGTCATAGTCCGCG.

Real-time PCR was performed using an ABI-Prism 7700 sequence detector (PE Applied Biosystems) and results were presented as Ct (the threshold cycle) values. The relative expression levels were calculated after normalization to the level of β-actin.

### EXAMPLE 5

### Immunoprecipitation and Immunoblotting.

293T cell were transfected with Flag-tagged TAZ and/or Myc-tagged MyoD expression vectors and harvested 2 days post-transfection. Whole cell extracts were incubated with Flag-M2 agarose beads (Sigma-Aldrich), which were then washed with lysis buffer. Immune complexes or whole cell extracts were resolved by SDS-PAGE and transferred to an Immobilon-P membrane (Millipore, Invitrogen). Blots were incubated with antibodies against MyoD, myogenin, myosin heavy chain (MHC), MEF2C, TAZ and -actin (Santa Cruz Biotechnology, Santa Cruz, Calif., USA).

### EXAMPLE 6

### Immunofluorescence Analysis

C2C12 cells were fixed and permeabilized with 2% formaldehyde and 0.1% Triton X-100. Cells were loaded for 20 min with antibodies to myogenin, MyoD and TAZ and subsequently incubated with Alexa Fluor 488- or Alexa Fluor 555-conjugated secondary antibodies (Molecular Probes, Invitrogen). Nuclei were counterstained using 4′,6-diamidino-2-phenylindole, dihydrochloride (DAPI) in mounting solution.

### EXAMPLE 7

### Electrophoretic Mobility Shift Assay (EMSA)

Nuclear proteins were prepared from 293T cells that were transfected with MyoD and/or TAZ expression vectors. 10 of nuclear proteins were incubated with radiolabeled double-stranded DNA in reaction buffer (10 mM Tris, pH 8.0, 150 mM KCl, 0.5 mM EDTA, 0.1% Triton X-100, 12.5% glycerol, 0.2 mM DTT). The DNA-protein complexes were resolved by 4% non-denaturing PAGE and visualized by autoradiography. The E-box site within MCK promoter was synthesized, annealed and labeled with radioisotope. The E-box site of MCK was as follows:

MCK-E-TOP
(SEQ ID No. 13)
5′-CCCCCCAACACCTGCTGCCTGAGC-3′;
MCK-E-Bottom
(SEQ ID No. 14)
5′-GGCTCAGGCAGCAGGTGTTGGGGG-3′.

### EXAMPLE 8

### Chromatin Immunoprecipitation

C2C12 cells were treated with 1% formaldehyde in order to cross-link protein complex to DNA and incubated for 10 min at 37° C. Cells were harvested, resuspended in 0.1% SDS-containing lysis buffer (50 mM HEPES, pH7.5, 140 mM NaCl, 1 mM EDTA, pH 8.0, 1% Triton X-100, 0.1% Sodium Deoxycholate, 0.1% SDS) and sonicated on ice to produce genomic DNA fragments. Supernatants were then incubated with anti-MyoD, anti-Flag or control antibodies at 4° C. overnight, followed by incubation with protein A/G-agarose beads (Pierce, Rockford, Ill., USA).

Immune complexes were washed three time with low-salt wash buffer (0.1% SDS, 1% Triton X-100. 2 mM EDTA, 150 mM NaCl, 20 mM Tris), twice with high-salt wash buffer (0.1% SDS, 1% Triton X-100, 2 mM EDTA, 500 mM NaCl, 20 mM Tris) and then eluted with 1% SDS and 0.1M NaHCO₃ by heating at 67° C. for 4 h to reverse formaldehyde cross-links. DNA was recovered using the PCR Purification kit (Qiagen, Hilden, Germany) and then used for PCR. Primers were as follows:

myogenin-CHIP-FWD
(SEQ ID No. 15)
5′-GAATCACATGTAATCCACTGG-3′,
myogenin-CHIP-REV
(SEQ ID No. 16)
5′-CACACCAACTGCTGGGTGCCA-3′..

### EXAMPLE 9

### Skeletal Muscle Injury in Mice

C57BL/6 mice were purchased from the Jackson Laboratories (Bar Harbor, Me., USA) and housed in Ewha Laboratory Animal Genomic Center under specific pathogen-free conditions. Mice were lightly anesthetized with sodium pentobarbital (Sigma-Aldrich) and a 1.5 cm-long incision was made through aseptically prepared skin overlaying the right tibialis anterior (TA) muscle.

A sterile stainless steel needle was pre-cooled in liquid nitrogen and inserted into TA muscle belly for 10 sec, as reported. Mice were sacrificed at the indicated time points after injury for the preparation of protein extracts and tissue sections. All animal experiments were performed with the approval of the Ewha Womans University Institutional Animal Care and Use Committee.

### EXAMPLE 10

### Immunohistochemistry

Injured TA muscles and control muscle were collected and fixed in 4% paraformaldehyde. Muscles were embedded in Tissue Tek OCT (Optimal Cutting Temperature solution, Miles Scientific, Elkart, Ind., USA), frozen in melting isopentane and stored at −80 ° C. Muscles were sectioned with 10 thickness on a motorized microtome in a cryostat (Leica RM 2255, Germany) and then incubated with anti-TAZ Ab (Abcam Inc., Cambridge, Mass., USA). Staining was developed with a DAB (diaminobenzidine) staining kit (R&D Systems, Minneapolis, Minn., USA). Slides were observed under a histology microscope (Eclipse E2000, Nikon, Japan).

### EXAMPLE 11

### Statistical Analysis

The results were given as mean ±SEM. The data were accumulated from at least three independent experiments. Statistical significance was determined by one-way ANOVA or two-tailed unpaired Student's t-test. P value less than 0.05 (P<0.05) was considered statistically significant.

### REFERENCES

1. Kanai, F., Marignani, P. A., Sarbassova, D., Yagi, R., Hall, R. A., Donowitz, M., Hisaminato, A., Fujiwara, T., Ito, Y., Cantley, L. C., and Yaffe, M. B. (2000) TAZ: a novel transcriptional co-activator regulated by interactions with 14-3-3 and PDZ domain proteins. EMBO J 19, 6778-6791
2. Cui, C. B., Cooper, L. F., Yang, X., Karsenty, G., and Aukhil, I. (2003) Transcriptional coactivation of bone-specific transcription factor Cbfal by TAZ. Mol Cell Biol 23, 1004-1013
3. Hong, J. H., Hwang, E. S., McManus, M. T., Amsterdam, A., Tian, Y., Kalmukova, R., Mueller, E., Benjamin, T., Spiegelman, B. M., Sharp, P. A., Hopkins, N., and Yaffe, M. B. (2005) TAZ, a transcriptional modulator of mesenchymal stem cell differentiation. Science 309, 1074-1078
4. Di Palma, T., D′Andrea, B., Liguori, G. L., Liguoro, A., de Cristofaro, T., Del Prete, D., Pappalardo, A., Mascia, A., and Zannini, M. (2009) TAZ is a coactivator for Pax8 and TTF-1, two transcription factors involved in thyroid differentiation. Exp Cell Res 315, 162-175
5. Park, K. S., Whitsett, J. A., Di Palma, T., Hong, J. H., Yaffe, M. B., and Zannini, M. (2004) TAZ interacts with TTF-1 and regulates expression of surfactant protein-C. J Biol Chem 279, 17384-17390
6. Murakami, M., Nakagawa, M., Olson, E. N., and Nakagawa, 0. (2005) A WW domain protein TAZ is a critical coactivator for TBX5, a transcription factor implicated in Holt-Oram syndrome. Proc Natl Acad Sci U S A 102, 18034-18039
7. Murakami, M., Tominaga, J., Makita, R., Uchijima, Y., Kurihara, Y., Nakagawa, 0., Asano, T., and Kurihara, H. (2006) Transcriptional activity of Pax3 is co-activated by TAZ. Biochem Biophys Res Commun 339, 533-539
8. Kang, H. S., Beak, J. Y., Kim, Y. S., Herbert, R., and Jetten, A. M. (2009) Glis3 is associated with primary cilia and Wwtrl/TAZ and implicated in polycystic kidney disease. Mol Cell Biol 29, 2556-2569
9. Kitagawa, M. (2007) A Sveinsson's chorioretinal atrophy-associated missense mutation in mouse Teadl affects its interaction with the co-factors YAP and TAZ. Biochem Biophys Res Commun 361, 1022-1026
10. Mahoney, W. M., Jr., Hong, J. H., Yaffe, M. B., and Farrance, I. K. (2005) The transcriptional co-activator TAZ interacts differentially with transcriptional enhancer factor-1 (TEF-1) family members. Biochem J 388, 217-225
11. Varelas, X., Sakuma, R., Samavarchi-Tehrani, P., Peerani, R., Rao, B. M., Dembowy, J., Yaffe, M. B., Zandstra, P. W., and Wrana, J. L. (2008) TAZ controls Smad nucleocytoplasmic shuttling and regulates human embryonic stem-cell self-renewal. Nat Cell Biol 10, 837-848
12. Chan, S. W., Lim, C. J., Guo, K., Ng, C. P., Lee, I., Hunziker, W., Zeng, Q., and Hong, W. (2008) A role for TAZ in migration, invasion, and tumorigenesis of breast cancer cells. Cancer Res 68, 2592-2598
13. Yun, K., and Wold, B. (1996) Skeletal muscle determination and differentiation: story of a core regulatory network and its context. Curr Opin Cell Biol 8, 877-889
14. Rudnicki, M. A., Schnegelsberg, P. N., Stead, R. H., Braun, T., Arnold, H. H., and Jaenisch, R. (1993) MyoD or Myf-5 is required for the formation of skeletal muscle. Cell 75, 1351-1359
15. Bischoff, R., and Heintz, C. (1994) Enhancement of skeletal muscle regeneration. Dev Dyn 201, 41-54
16. Hasty, P., Bradley, A., Morris, J. H., Edmondson, D. G., Venuti, J. M., Olson, E. N., and Klein, W. H. (1993) Muscle deficiency and neonatal death in mice with a targeted mutation in the myogenin gene. Nature 364, 501-506

What is claimed is:

1. A method for promoting muscle differentiation or regeneration, comprising administering an enhancer capable of inducing or promoting the binding of TAZ polypeptide with MyoD polypeptide to a subject or muscle cell thereof in need of muscle differentiation or regeneration.

2. The method according to claim 1, wherein the method further comprises administering a TAZ polypeptide, a fragment thereof, or a polynucleotide encoding the same; a MyoD polypeptide, a fragment thereof, or a polynucleotide encoding the same; or a mixture thereof.

3. The method according to claim 1, wherein the enhancer inhibits the activity of an inhibitor that suppresses or inhibits the binding of TAZ polypeptide with MyoD polypeptide.

4. A method for inhibiting muscle differentiation or regeneration, comprising administering an inhibitor capable of suppressing or inhibiting the binding of TAZ polypeptide with MyoD polypeptide to a subject or muscle cell thereof in need of inhibiting muscle differentiation or regeneration.

5. A method for screening a substance capable of up- or down-regulating muscle differentiation or regeneration by use of interaction between TAZ polypeptide and MyoD polypeptide.

6. The method according to claim 5, wherein the interaction is a direct or indirect binding of TAZ polypeptide with MyoD polypeptide.

7. An isolated peptide consisting of an amino acid sequence of SEQ ID NO. 2, a fragment thereof, or a variant of SEQ ID NO. 2 having 60% or more homology therewith, wherein said peptide binds to and activates the MyoD polypeptide.

8. The peptide according to claim 7, wherein the isolated peptide has an activity of inducing muscle differentiation or regeneration.

9. The peptide according to claim 7, wherein the isolated peptide is an amino acid region at positions 124-395 of TAZ polypeptide of SEQ ID NO. 1.

10. A polynucleotide encoding the isolated peptide of claim 7.

11. The polynucleotide according to claim 10, wherein the polynucleotide is in a form of expression vector.

12. A pharmaceutical composition comprising the isolated peptide of claim 7 or the polynucleotide encoding the same.

13. The pharmaceutical composition according to claim 12, wherein the pharmaceutical composition is used for inducing muscle differentiation or regeneration, or treating muscular disease.

14. The pharmaceutical composition according to claim 13, wherein the muscular disease is skeletal muscle disorders, cardiac muscle pathologies, or smooth muscle disorders.

15. The pharmaceutical composition according to claim 13, wherein the muscular disease is selected from the group consisting of myopathies, myoneural conductive diseases, traumatic muscle injury, nerve injury, ischemic damage, congenital and traumatic disorders, arterial sclerosis, vascular lesions, and congenital vascular diseases.
